(19) United States
(12) Patent Application Publication
(10) Pub. No.: US 2005/0089608 A1
(43) Pub. Date: Apr. 28, 2005

(54) Biological method of obtaining a food preparation with a base of haem iron, as well as the food preparation obtained by implementing the method

(76) Inventors: Fouzia Nedjaoum, Reims (FR); Pascal Dhulster, Villeneuve D'Ascq (FR); Nouria Fatah, Vendeville (FR); Didier Guillochon, Marcq en Baroeuil (FR); Charles LeGrand, Caen (FR); Marie-Pascale Lepley-LeGrand, Caen (FR)

Correspondence Address: BAKER & DANIELS;111 E. WAYNE STREET, SUITE 800, FORT WAYNE, IN, 46802, US

(21) Appl. No.: 10/847,713

(22) Filed: May 18, 2004

(30) Foreign Application Priority Data
Jun. 4, 2003 (FR) 03 06 720

(51) Int. Cl. A61K 38/16

(52) U.S. Cl. 426/264

(57) ABSTRACT

Method characterised by the following steps: a first fraction of animal's blood, for example pig's blood or cow's blood or alternatively derivatives thereof such as cruor, is subjected to a process of controlled enzyme hydrolysis to obtain a first hydrolysate containing haem iron complexed with large-sized peptides; the first hydrolysate is enriched by ultra-filtration to obtain a hydrolysate solution enriched with haem iron-peptide complexes; in parallel, a second fraction of animal's blood or derivatives thereof such as cruor, is subjected to a process of advanced enzyme hydrolysis to obtain an extrinsic haem solution highly complexed with small peptides; the hydrolysate solution enriched with haem iron-peptide complexes is mixed with the extrinsic haem solution to enable the extrinsic haem to fix on the large-sized peptides of the hydrolysate solution and to obtain a haemic peptide fraction with a high content of iron soluble at an acid pH.

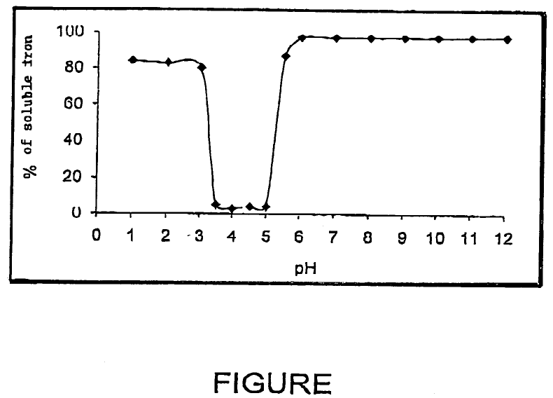

The present invention relates to a food preparation with a high content of haem iron-peptide complexes soluble in an acid medium, for the prevention and treatment of iron deficiency, as well as a biological process enabling this preparation to be obtained.

Iron deficiency is a major health problem and is common in the industrialised countries and developing nations alike, affecting approximately 20% of the world's population.

In quantitative terms, iron is the most important trace element in the organism and is indispensable for maintaining cellular life. It is particularly essential to the transport of oxygen and electrons as well as DNA synthesis and is an active element in numerous molecules which play a vital role in a broad range of cellular functions, including haemoglobin (which carry oxygen), cytochromes (electron carriers in the respiratory tracts) and haem enzymes (catalase, peroxydase).

The recommended intake of iron (mg per day) is as follows:

| | |
|---|---|
| 6-12 months | 7 |
| 1-3 years | 7 |
| 4-9 years | 7 |
| 10-12 years | 8 |
| Adolescent males aged 13-19 years | 12 |
| Adolescent females aged 13-19 years | 14 |
| Adult males | 9 |
| Menstruating females | 16 |
| Menopausal females | 9 |
| Breast-feeding females | 10 |
| Pregnant females | 25-35 |

Iron originating in food exists in two forms, which are absorbed by the enterocytes of jejunum by a different absorption mechanism: non-haemic iron and haem iron.

Haem iron is present in two food proteins, haemoglobin and myoglobin, primarily found in red meat.

Haem is made up of an iron atom at the centre of a non-protein, organic, flat cyclic structure containing four pyrrole nuclei joined by methenyl bridges as well as eight lateral chains.

Absorption of haem iron (20 to 30%) is largely superior to that of non-haemic iron because it is not greatly influenced by dietary factors and gastric secretions.

Non-haemic iron constitutes approximately 90% of the iron present in food: in effect, 60% of iron of animal origin and all iron of vegetable iron is non-haemic; it is found in cereals, dried legumes, fruit, dairy products and vegetables.

The amount of non-haemic iron absorbed is lower than that of haem iron (2 to 20%) and varies with the composition of the diet. It may be stimulated by certain dietary factors or, conversely, inhibited by others.

Nowadays, specialists offer various treatments for iron deficiency (change of diet, the addition of iron to food or alternatively iron supplements).

A whole range of preparations based on iron salts are currently available on the market (iron sulphate, iron gluconate, iron fumarate, etc.), as a means of treating and preventing iron deficiency.

However, this source of non-haemic iron comes with a number of disadvantages, including the following:
its absorption varies with the composition of the diet
poor digestive tolerance (the astringent nature of iron salts causes intestinal problems in 20% of cases, which means that treatment cannot be continued)
an oxidising action (which risks generating free radicals in situ in the colon, the harmful effects of which have already been demonstrated).

The objective of the invention is to propose a food preparation specifically adapted to the treatment and prevention of iron deficiency which will remedy these drawbacks and exhibit an excellent bio-availability.

It has been discovered that haem iron is the form of iron which can be most readily assimilated and causes fewer undesirable effects than mineral iron.

Haemoglobin is an important source of haem iron, given that it is an easy sub-product to obtain and is inexpensive.

For dietary reasons, however, it is inconceivable to use haemoglobin in supplements because it contains a proportion of only 0.34% by weight of iron, the rest being proteins.

Consequently, administering haemoglobin in therapeutic doses would be likely to cause an imbalance in protein levels, particularly in industrialised countries where diets are already very rich in proteins, in some cases even too rich: in practice, it would be necessary to ingest 6 g of pure haemoglobin in order to satisfy the recommended daily intake of iron.

In view of this situation, it would be desirable to find preparations with a haem iron base for treating iron deficiency, derived from haemoglobin with an iron content significantly in excess of 0.34% (w/w).

Numerous methods have already been proposed as a means of obtaining such preparations.

To date, however, none of these methods has proved totally satisfactory due to problems of bio-availability.

In order to increase the iron content of haemoglobin, some teams have separated haem from globin with methods using organic solvents, all of which have resulted in preparations with high concentrations of haem iron. However, absorption of the haem iron in these preparations is limited.

According to a publication by Uzel C. and Conrad M. E. (1998) entitled Absorption of heme iron, Semin. Hematol. 35, 27-34, it was found from studies conducted amongst males that the absorption of haem is very poor if administered separately from globin. It was found that when not transported by globin, haem forms aggregates which precipitate on exposure to the acid pH of the stomach and are not absorbed by the intestine. It would appear that the products resulting from the break-down of haemoglobin, i.e. peptides, play a role in the absorption of haem iron by complexing it, thereby limiting its aggregation.

According to the publication by Schümann K., Elsenhans B. and Mäurer A. (1998) entitled Iron supplementation, J. Trace Elements Med. Biol. 12, 129-140, adding whole proteins when administering haem appears to increase bio-availability.

These different studies have therefore established that the bio-availability of haem iron seems to be linked to the fact that it is in a complexed state: isolated haem iron forms aggregates which are insoluble at the acid pH of the stomach and is therefore not absorbed by the intestine, whereas haem iron complexed with proteins (as in the case of haemoglobin) or peptides forms fewer aggregates and is totally soluble at the acid pH of the stomach; it is therefore absorbed.

In view of this situation, specialists have attempted to come up with preparations based on a haem complexed with peptides or proteins in order to render it soluble at the acid pH prevailing in the stomach.

Accordingly, several teams have developed methods combining enzymatic hydrolysis of haemoglobin followed by ultra-filtration of the peptide hydrolysates resulting from this hydrolysis with a view to preparing fractions of haemic peptides with a higher iron concentration than haemoglobin.

To date, however, no process has been proposed which enables fractions of haemic peptides with a high ratio of haem iron/peptides to be prepared and in which haem is complexed with peptides of a large size which limit its state of aggregation and enable it to be rendered soluble at an acid pH.

By way of example, according to the publication by Eriksson C. (1982) entitled Heme-iron enriched amino acid and a process for the preparation of heme-iron-enriched amino acid, and document U.S. Pat. No. 4,411,915, a preparation process using the blood of animals, for example pig's blood or cow's blood, or alternatively its derivatives such as cruor, has been proposed as a means of obtaining a composite preparation of haem and short peptide sequences in which the iron/peptide ratio is up to fifteen times higher than that of haemoglobin.

This iron-enriched preparation is the result of a process of advanced hydrolysis of denatured haemoglobin. Enrichment is obtained by centrifugation or ultra-filtration or by combining centrifugation and ultra-filtration.

However, the haem present in this fraction is in a very highly aggregated form, which would limit its absorption. In effect, the haem is complexed with peptides of a very small size, which are not capable of preventing precipitation of these large haem aggregates at an acid pH.

Although it has proved possible to obtain a high degree of iron enrichment compared with haemoglobin (up to 15 times under certain conditions), this iron is not potentially bio-available because it is complexed with small-sized peptides which are incapable of preventing its precipitation at an acid pH.

In another example, according to document KR 10-2000-0053778 and the publication in M. J. by Chae, H. J. and Oh, N. S. (2002) entitled Process development for heme-enriched peptide by enzymatic hydrolysis of hemoglobin, Biores. Technol. 84, 63-68, another method of preparing a peptide fraction with a high iron content from haemoglobin has been described.

This method of preparation is based on a very advanced hydrolysis of haemoglobin by admixing proteases and ultra-filtration.

The enrichment factor obtained with this method may be up to 14.

Unfortunately, the haem iron in this preparation is bonded to small-sized peptides which are incapable of preventing the formation of aggregates and hence maintaining it in solution in an acid medium.

According to the publication by Lebrun F., Bazus A., Dhulster P. and Guillochon D, (1998), entitled Influence of molecular interactions on ultrafiltration of a bovine hemoglobin hydrolysate with an organic membrane, J. Membr. Sci. 146, 113-124, a method has been described which enables a hydrolysate of haemic peptides to be obtained from pig's blood or cow's blood or alternatively its derivatives such as cruor, which contains 2.5 times more iron than haemoglobin and in which the haem is bonded to peptides of a large size.

The preparation obtained by this method contains haem-peptide complexes which are soluble in a broad range of pH, including the acid pH range; these large-sized peptides have the same capacity as the globin in haemoglobin to limit aggregation of the haem and to transport it in an acid medium.

However, the degree of iron enrichment obtained is totally inadequate.

Finally, according to the publication by Seligman P. A., Moore G. M. and Rhoda B. S. (2000) entitled Clinical studies of HIP: an oral heme-iron product, Nutr. Res. 20, 1279-1286, a preparation based on haem iron containing a proportion of 1% of iron, in other words three times that of native haemoglobin, has been tested on humans but only half is soluble at an acid pH. This preparation was found to exhibit a bio-availability in humans that is very much higher than that of the concentrated haem.

Unfortunately, all the methods described above result in preparations which are either rich in haem iron but the haem iron is insoluble at an acid pH, or preparations in which the haem iron is soluble but the iron content is not high enough.

Against this background, the objective of the invention is to propose a food preparation with a base of haem iron which is potentially bio-available, specifically adapted for the treatment of iron deficiency, intended to fill this gap.

Accordingly, the present invention relates to a method of preparing a fraction of haemic peptides with a high content of haem iron, in which the majority of haem iron is complexed with peptides of a large size.

In view of the fact that this preparation is intended for use as a dietary complement, it is essential that the method should be a bio-process which does not use a solvent.

As proposed by the invention, this method is characterised by the following successive steps:
a first fraction of animal's blood, for example pig's blood or cow's blood or alternatively derivatives thereof such as cruor, is subjected to a process of controlled enzyme hydrolysis to obtain a first hydrolysate containing haem iron complexed with large-sized peptides as well as populations of small-sized peptides not complexed with the haem iron;
the first hydrolysate thus obtained is enriched to obtain haem iron-peptide complexes by ultra-filtration in order to remove peptide populations not complexed with the haem iron and to obtain a hydrolysate solution enriched with haem iron-peptide complexes in which the haem iron is complexed with large-sized peptides, thereby rendering it soluble at an acid pH;
in parallel, a second fraction of animal's blood, for example pig's blood or cow's blood or alternatively derivatives thereof such as cruor, is subjected to a process of advanced enzyme hydrolysis to obtain a second hydrolysate containing haem iron complexed with small peptides as well as populations of small-sized peptides not complexed with the haem iron;
the second hydrolysate thus obtained is centrifuged in order to remove a large proportion (approximately 60%) of the small peptides not bonded to the haem and obtain a centrifuged residue with a very high haem concentration;
the centrifuged residue is rendered soluble, in particular by adding soda, to obtain an extrinsic solution of haem in which the haem is bonded to small-sized peptides and is therefore totally insoluble at an acid pH;
the hydrolysate solution enriched with haem iron-peptide complexes is mixed with the extrinsic haem solution to enable the extrinsic haem to fix on the large-sized peptides of the enriched hydrolysate solution and release the small peptides bonded to the extrinsic haem;
the resultant mixture is centrifuged in order to remove the extrinsic haem which has not fixed to the large-sized peptides; and
the resultant solution is enriched with haem iron-peptide complexes by ultra-filtration in order to remove the small peptides released during the mixing step.

The method proposed by the invention is therefore based on a particular property of certain haem iron-peptide complexes which are capable of fixing additional quantities of haem.

This method therefore involves subjecting two fractions of animal's blood, for example pig's blood or cow's blood or alternatively derivatives thereof such as cruor, to a process of enzyme hydrolysis in parallel, namely, on the one hand, controlled hydrolysis of a first fraction to obtain a peptide fraction in which the haem iron is complexed with peptides of a large size which will subsequently render it soluble at an acid pH, and, on the other hand, advanced hydrolysis of a second fraction, enabling a peptide fraction containing small-sized peptides to be obtained, some of which are complexed with haem.

The advanced hydrolysis is followed by a step during which approximately 60% of the small peptides not complexed with haem are removed by centrifugation in order to obtain a peptide fraction with a high concentration of haem iron (extrinsic haem solution). This haem iron is then fixed on the large-sized peptides obtained from the controlled hydrolysis.

It would appear that the extrinsic haem is fixed on the large-sized peptides due to hydrophobic interactions.

It should be pointed out that the extrinsic haem is totally insoluble at an acid pH but is subsequently rendered soluble due to its attachment to the large peptides.

After purification, this method enables a food preparation to be obtained, which has a very high content of iron compared with haemoglobin, which is soluble between pH 1 and pH 3 and between pH 5.5 and pH 12, and in which 80% of the iron is complexed with peptides and hence potentially bio-available.

For the purposes of the invention, the fraction of haemic peptides with an ultimately high concentration of iron may be prepared in liquid form but is advantageously subjected to a drying step to obtain a food preparation in the form of a powder, suitable for the treatment of iron deficiency.

Such a powder may advantageously be incorporated in capsules so that they can be absorbed as a dietary complement.

The drying process used for the purposes of the invention (vacuum oven and grinding, atomisation or freeze-drying and grinding) imparts the properties (flow capacity, dispersion capacity, etc.) of the powders ultimately obtained.

It has proved to be more advantageous to use a vacuum oven for drying purposes followed by grinding in situations where the powder is subsequently to be introduced into capsules.

For the purposes of the invention, the initial controlled enzyme hydrolysis and the advanced enzyme hydrolysis of the two fractions of animal's blood, for example pig's blood or cow's blood or alternatively derivatives thereof such as cruor, are preferably conducted using an enzyme that is active at an acid pH, in particular porcine pepsin.

It should be pointed out that porcine pepsin has a number of advantages.

In effect, it is a digestive enzyme which permits hydrolysis at an acid pH, thereby preventing bacterial contamination. Furthermore, the enzyme reaction can easily be halted at the desired stage by raising the pH to 8 again (pH at which pepsin is denatured), in particular by adding diluted soda.

In accordance with the invention, it has been found that the controlled enzyme hydrolysis reaction and the advanced enzyme hydrolysis reaction are best operated at a pH of less than 6.

The pH of animal's blood, for example pig's blood or cow's blood or alternatively derivatives thereof such as cruor, may be adjusted to the desired value by adding hydrochloric acid.

By way of example, if the initial raw material is cruor, the degree of hydrolysis during the controlled enzyme hydrolysis step is adjusted to a value of less than or equal to 5%, whereas the degree of hydrolysis for the advanced enzyme hydrolysis step is adjusted to 15% and more.

The degree of hydrolysis corresponds to the percentage of peptide bonds which are cut; specialists are perfectly familiar with the way in which this parameter is controlled, in particular by measuring the released NH₂ molecules.

The choice of degrees of hydrolysis is a decisive characteristic of the method proposed by the invention.

In practice, it has been found that below a certain size, peptides are no longer capable of rendering haem soluble at an acid pH or of fixing additional quantities of extrinsic haem.

Consequently, during the controlled hydrolysis, it is imperative to ensure that a sufficiently high iron enrichment is achieved in the first fraction of animal's blood, for example pig's blood or cow's blood or alternatively derivatives thereof such as cruor, whilst taking care not to decrease the size of the peptides used to complex the haem to too great a degree.

During the advanced hydrolysis, it is imperative to obtain a haem that is sufficiently pure but nevertheless bonded to small-sized peptides, which will enable it to be fixed onto the large-sized peptides in the fraction obtained by controlled hydrolysis, thereby preventing too high a degree of aggregation which could be detrimental to this fixing process.

In accordance with one preferred characteristic of the invention, during the step of mixing the hydrolysate solution enriched with haem iron-peptide complexes and the extrinsic haem solution, the quantities of solutions used are such that the quantity of haem contributed by the extrinsic haem solution is up to ten times higher than the quantity of haem in the hydrolysate solution.

For the purposes of the invention, the two solutions are mixed at ambient temperature, accompanied by agitation as a general rule.

The step of enrichment by ultra-filtration then enables the small peptides released during the mixing step to be removed.

By virtue of another feature of the invention, hydrophilic membranes with a cut-off threshold of approximately 10 kDa are used for the ultra-filtration enrichment steps.

It has been found that using such membranes enables peptides that are not complexed with haem to be removed at a haem retention rate of close to 100%.

For the purposes of the invention, it is of particular advantage to run at least one, preferably three, dia-filtration steps on identical membranes after each of the ultra-filtration enrichment steps, in order to increase enrichment of the hydrolysate whilst continuing to remove peptides.

Tests have shown that no further enrichment to speak of is obtained over and above three dia-filtration steps.

In accordance with the invention, it is also of advantage to centrifuge the first hydrolysate before enriching it by ultra-filtration in order to remove insoluble peptides which accumulate in the centrifuged residue.

Tests have shown that this centrifugation does not lead to a loss of haem and in particular lengthens the time before the ultra-filtration membranes become clogged.

For the first time, the invention offers a method enabling a food preparation with a haem base to be obtained from animal's blood, for example pig's blood or cow's blood or alternatively derivatives thereof such as cruor which contains both:
a high quantity of iron which may be up to ten times higher than that of a haemoglobin preparation; and
a haem iron in a form that can be readily assimilated.

Furthermore, the invention also proposes, for the first time, a method which enables enzymatic hydrolysates of animal's blood, for example pig's blood or cow's blood or alternatively derivatives thereof such as cruor, to be enriched with haem iron which is more readily assimilated by a factor of up to ten.

The invention further relates to a food preparation with a base of haem iron specifically adapted for the treatment of iron deficiency and in particular obtained by implementing the method described above.

Such a food preparation may be produced in liquid form or in the form of a powder of haemic peptides with an iron/peptide weight ratio in excess of 2%, preferably approximately 3%, in which more than 80% of this iron is complexed with the peptides.

Such a powder is soluble at the acid pH prevailing in the stomach.

Consequently, the invention offers a powdered dietary complement exhibiting a good bio-availability which is, moreover, very highly enriched with iron as compared with haemoglobin.

By virtue of one particularly advantageous characteristic of the invention, the weight by volume of this powder when compressed is close to 1.

The invention further relates to a food preparation in the form of capsules, with, for example, a capacity by volume of approximately 0.5 ml, filled with powdered haemic peptides.

Such capsules may advantageously be gastro-protected.

This format enables the preparation of haemic peptides to be protected against uncontrolled peptic hydrolysis in the stomach.

Such hydrolysis processes would effectively lead to the formation of small-sized peptides and amino acids, which are incapable of rendering the haem aggregates soluble at the acid pH prevailing in the stomach.

The invention therefore offers consumers the recommended daily-intake of iron, for which purpose it will be necessary to absorb three capsules containing approximately 0.5 ml per day, which corresponds to an acceptable formulation.

The fact that the method proposed by the invention is followed by a step of drying the fraction of haemic peptides with a high iron content in a vacuum oven, for example, enables a powder to be obtained, the physical-chemical characteristics of which are set out in the table below.

| | |
|---|---|
| % iron (w/w) | 2.1 |
| % complexed iron (w/w) | 1.7 |
| % peptides (w/w) | 75 |
| ratio iron/peptides % (w/w) | 2.8 |
| weight by volume compressed | 0.80 |

The solubility curve of the iron in this fraction of dried haemic peptides as a function of pH is set out in the appended diagram.

To obtain this curve, 0.5%-strength solutions (w/v) were prepared by diluting the powder in buffer solutions with a pH ranging from 1 to 12. These solutions were centrifuged and the quantity of iron present in the supernatant was measured. This curve demonstrates that the iron in this peptide fraction is readily soluble in the pH acid range (pH 1 to pH 3), thus confirming that the peptides carry the haem. It should be pointed out that the iron is not soluble within a pH range of between 3.5 and 5. This precipitation zone corresponds to the pH of the peptides. On precipitation, the latter entrain with them the haem which they carry.

1) Biological method of obtaining a food preparation with a base of haem iron, specifically adapted for the treatment of iron deficiency, from animal's blood, for example pig's blood or cow's blood or alternatively derivatives thereof such as cruor, characterised by the following successive steps:
a first fraction of animal's blood is subjected to a process of controlled enzyme hydrolysis to obtain a first hydrolysate containing haem iron complexed with large-sized peptides as well as populations of peptides not complexed with the haem iron;
the first hydrolysate thus obtained is enriched to obtain haem iron-peptide complexes by ultra-filtration in order to remove peptide populations not complexed with the haem iron and to obtain a hydrolysate solution enriched with haem iron-peptide complexes;
in parallel, a second fraction of animal's blood is subjected to a process of advanced enzyme hydrolysis to obtain a second hydrolysate containing haem iron complexed with small peptides as well as populations of peptides not complexed with the haem iron;
the second hydrolysate thus obtained is centrifuged in order to remove a large proportion of the small peptides not bonded to the haem and obtain a centrifuged residue with a very high haem concentration;
the centrifuged residue is rendered soluble, in particular by adding soda, to obtain an extrinsic solution of haem in which the haem is bonded to small-sized peptides;
the hydrolysate solution enriched with haem iron-peptide complexes is mixed with the extrinsic haem solution to enable the extrinsic haem to fix on the large-sized peptides of the hydrolysate solution and release the small peptides bonded to the extrinsic haem;
the resultant mixture is centrifuged in order to remove the extrinsic haem which has not fixed to the large-sized peptides; and
the resultant solution is enriched with haem iron-peptide complexes by ultra-filtration in order to remove the small peptides released during the mixing step.

2) Method as claimed in claim 1, characterised in that the fraction of haemic peptides with a high iron content is dried to obtain a food preparation in the form of a powder, specifically adapted for the treatment of iron deficiency.

3) Method as claimed in claim 1, characterised in that the initial controlled enzyme hydrolysis and the advanced enzyme hydrolysis of the two fractions of animal blood are conducted with an enzyme that is active at an acid pH, in particular porcine pepsin.

4) Method as claimed in claim 3, characterised in that the controlled enzyme hydrolysis and the advanced enzyme hydrolysis are conducted at a pH of less than 6.

5) Method as claimed in claim 1, characterised in that if the initial raw material is cruor, the degree of hydrolysis during the controlled enzyme hydrolysis step is adjusted to a value of less than or equal to 5%, whereas the degree of hydrolysis for the advanced enzyme hydrolysis step is adjusted to at least 15%.

6) Method as claimed in claim 1, characterised in that during the step of mixing the hydrolysate solution enriched with haem iron-peptide complexes and the extrinsic haem solution, the quantities of solutions used are such that the quantity of haem contributed by the extrinsic haem solution is up to ten times higher than the quantity of haem in the hydrolysate solution.

7) Method as claimed in claim 1, characterised in that hydrophilic membranes with a cut-off threshold of approximately 10 kDa are used for the ultra-filtration enrichment steps.

8) Method as claimed in claim 1, characterised in that one, preferably three, dia-filtration steps on identical membranes are run after each of the ultra-filtration enrichment steps, in order to increase enrichment of the hydrolysate whilst continuing to remove peptides.

9) Method as claimed in claim 1, characterised in that the first hydrolysate is centrifuged in order to remove insoluble peptides which accumulate in the centrifuged residue before enriching it by ultra-filtration.

10) Food preparation with a base of haem iron specifically adapted for the treatment of iron deficiency, obtained by implementing the method as claimed in claim 1, characterised in that it is produced in the form of a liquid or a powder of haemic peptides with an iron/peptide weight ratio in excess of 2%, preferably approximately 3%, in which more than 80% of this iron is complexed with the peptides.

11) Food preparation with a base of haem iron specifically adapted for the treatment of iron deficiency, obtained by implementing the method as claimed in claim 1, characterised in that it is prepared in the form of capsules, in particular with a capacity by volume of approximately 0.5 ml, filled with powdered haemic peptides as claimed in claim 10.

12) Food preparation as claimed in claim 11, characterised in that the gel capsules are gastro-protected.
